(19) United States
(12) Patent Application Publication
(10) Pub. No.: US 2023/0276836 A1
(43) Pub. Date: Sep. 7, 2023

(54) ORAL RETENTION COMPOSITION

(71) Applicant: BIZEN CHEMICAL CO., LTD., Akaiwa-shi, Okayama (JP)

(72) Inventors: Junichiro WAKAMATSU, Akaiwa-shi, Okayama (JP); Michiko MATSUMOTO, Akaiwa-shi, Okayama (JP); Hirotaka HIRANO, Akaiwa-shi, Okayama (JP); Hitomi WATANABE, Akaiwa-shi, Okayama (JP); Koki HAKATA, Akaiwa-shi, Okayama (JP); Keisuke URYU, Akaiwa-shi, Okayama (JP); Isafumi MARU, Akaiwa-shi, Okayama (JP)

(21) Appl. No.: 18/005,768

(22) PCT Filed: Jul. 20, 2021
(86) PCT No.: PCT/JP2021/027076

§ 371 Date: Jan. 17, 2023

(30) Foreign Application Priority Data
Jul. 20, 2020 (JP) 2020-123811

(51) Int. Cl. A23L 33/125; A23L 33/105; A23L 33/135

(52) U.S. Cl. CPC A23L 33/125; A23L 33/105; A23L 33/135

(57) ABSTRACT

An object of the present invention is to provide an oral retention composition in which a decrease in the viscosity of an acidic polysaccharide due to a functional substance is suppressed even when the functional substance is basic. The present invention relates to an oral retention composition comprising at least one compound selected from the group consisting of shellac and zein, a functional substance, and an acidic polysaccharide, the functional substance coated with the at least one compound selected from the group consisting of shellac and zein being mixed with the acidic polysaccharide.

### TECHNICAL FIELD

The present invention relates to an oral retention composition, and a composition for oral cavity or oral composition comprising the same. More specifically, the present invention relates to a composition with a long retention time in the oral cavity, and a composition for oral cavity or oral composition comprising the same.

### BACKGROUND ART

Functional substances are known to be applied in the oral cavity for the purpose of improving the oral environment (e.g., antibacterial (bactericidal and/or bacteriostatic) against resident oral bacteria, cariogenic bacteria, etc.). For example, protamine degradation products are known to have antifungal activity against Candida albicans, a resident oral bacterium (PTL 1).

However, functional substances are easily washed away in the oral cavity, which is constantly washed with saliva and exudate. For this reason, attempts have been made to retain functional substances in the oral cavity for a longer period of time in order to draw out more effects of the functional substances on the oral cavity. For example, it has been reported that a liquid oral care composition prepared by dissolving powder of catechin and xanthan gum in water gradually increased the catechin concentration upon contact with model saliva (PTL 2).

### CITATION LIST

### Patent Literature

PTL 1: JP2008-177679A
PTL 2: JP2007-016021A

### SUMMARY OF INVENTION

### Technical Problem

However, substances that are used in combination with functional substances (retention-enhancing substances) to improve the retention of the functional substances in the oral cavity may interact with the functional substances to greatly reduce or eliminate the effects of the functional substances on the oral cavity (PTL 1). Further, the present inventors realized that it was difficult to use acidic polysaccharides in oral retention compositions for the purpose of enhancing retention because when the functional substance was a basic substance or a polyphenol, the retention-enhancing action (e.g., viscosity-imparting) of the acidic polysaccharide was greatly reduced.

### Solution to Problem

The present inventors found that even when the functional substance is at least one member selected from the group consisting of basic substances and polyphenols, the decrease in the viscosity of the acidic polysaccharide caused by the functional substance can be suppressed, and the oral retention time becomes longer. The present invention has been completed based on these findings.

An object of the present invention is to provide a composition and a production method thereof set forth in the following items.

### Item 1.

An oral retention composition comprising at least one compound selected from the group consisting of shellac and zein, a functional substance, and an acidic polysaccharide, the functional substance coated with the at least one compound selected from the group consisting of shellac and zein being mixed with the acidic polysaccharide.

### Item 2.

The oral retention composition according to Item 1, wherein the acidic polysaccharide is at least one member selected from the group consisting of carrageenan, hyaluronic acid, xanthan gum, sodium alginate, pectin, and gum arabic.

### Item 3.

The oral retention composition according to Item 1 or 2, wherein the functional substance is at least one member selected from the group consisting of a basic substance, a polyphenol, and a lactic acid bacterium.

### Item 4.

The oral retention composition according to any one of Items 1 to 3, wherein the basic substance has an amino group, and the polyphenol has two or more hydroxyl groups bonded to an aromatic ring.

### Item 5.

The oral retention composition according to any one of Items 1 to 3, wherein the basic substance is at least one member selected from the group consisting of a protamine degradation product, arginine, lysine, glucosamine, spermine, spermidine, putrescine, piperine, and chitosan.

### Item 6.

The oral retention composition according to any one of Items 1 to 3, wherein the polyphenol is at least one member selected from the group consisting of chlorogenic acid, quercetin, resveratrol, tannic acid, caffeic acid, caffeic acid phenethyl ester, hydroxytyrosol, oleuropein, gallic acid, eugeniin, catechin, daidzein, procyanidin, theaflavin, polyphenol-containing plant extract, propolis, champignon extract, syringin, and eleutheroside E.

### Item 7.

The oral retention composition according to any one of Items 1 to 6, which is in the form of granules, powder, or tablets.

### Item 8.

The oral retention composition according to any one of Items 1 to 7, which has an average particle size of 1 µm to 400 µm.

### Item 9.

The oral retention composition according to any one of Items 1 to 8, which is a material for producing a composition for oral cavity or an oral composition.

### Item 10.

A composition for oral cavity or an oral composition, comprising the oral retention composition according to any one of Items 1 to 9.

### Item 11.

The composition for oral cavity or oral composition according to Item 10, which is a food, an oral care product, a pet food, a drug, or a quasi-drug.

### Item 12.

The composition for oral cavity according to Item 11, which is a food for bad breath control.

### Item 13.

The composition for oral cavity according to Item 12, which is a food for bad breath control while wearing a mask or a food for bad breath control at the time of awakening.

### Item 14.

The composition for oral cavity according to Item 11, which is a taste retention food or a taste-masking food.

### Item 15.

A method for producing an oral retention composition comprising at least one compound selected from the group consisting of shellac and zein, a functional substance, and an acidic polysaccharide, the functional substance coated with the at least one compound selected from the group consisting of shellac and zein being mixed with the acidic polysaccharide;

the method comprising:
step 1 of mixing the at least one compound selected from the group consisting of shellac and zein dissolved or dispersed in a solvent with the functional substance so that the compound coats the functional substance; and
step 2 of mixing the functional substance coated with the at least one compound selected from the group consisting of shellac and zein obtained in step 1 with the acidic polysaccharide.

### Item 16.

The production method according to Item 15, wherein in step 2, the functional substance is mixed with the acidic polysaccharide while ethanol or hydrous ethanol with an ethanol concentration of 50% to 99% (v/v) is added.

### Advantageous Effects of Invention

The present invention improves the oral retention of functional substances. Further, the present invention can suppress the action of functional substances to reduce the viscosity of acidic polysaccharides. The present invention can suppress the reaction between functional substances and acidic polysaccharides. The present invention can provide a method for producing an oral retention composition.

### BRIEF DESCRIPTION OF DRAWINGS

FIG. 1 is a graph showing the measurement results for absorbance in Test Example 8.

FIG. 2 is a graph showing the test results in Test Example 14.

FIG. 3 is a graph showing the test results in Test Example 15.

FIG. 4 is a graph showing the test results in Test Example 16.

### DESCRIPTION OF EMBODIMENTS

Unless otherwise specified, the symbols and abbreviations used in the present specification can be understood to have the meanings commonly used in the technical field to which the present invention pertains, in accordance with the context of the present specification.

In the present specification, the term “contain” is intended to include the terms “consist essentially of” and “consist of.”

Any step, treatment, or operation described in the present specification may be performed at room temperature, unless otherwise specified. In the present specification, room temperature can refer to a temperature of 10° C. to 40° C.

### Oral Retention Composition

The oral retention composition comprises (1) a functional substance, (2) at least one compound selected from the group consisting of shellac and zein (also referred to as “the coating compound” in the present specification), and (3) an acidic polysaccharide. The functional substance is coated with the coating compound, and the functional substance coated with the coating compound (also referred to as “the coated functional substance” in the present specification) is mixed with the acidic polysaccharide to form an oral retention composition.

The oral retention composition may have the function of retaining the functional substance in the oral cavity longer than when an acidic polysaccharide is not used and when the functional substance is not coated with a coating compound. Therefore, the oral retention composition can be used to retain the functional substance in the oral cavity longer so that the functional substance exerts its effects in the oral cavity for a long period of time.

### Functional Substance

The functional substance is a substance that can have intraoral functionality, i.e., the property of exerting its function on the living body by being present in the oral cavity. In the oral retention composition of the present invention, the functional substance is coated with a coating compound, and the coated functional substance is mixed with an acidic polysaccharide, whereby it can be retained in the oral cavity for a longer period of time, can exert its function for a longer period of time, and does not significantly impair the retention-imparting function of the acidic polysaccharide. The functional substances can be used singly or in combination of two or more.

The functional substance may be, for example, a substance that has the function of preventing or suppressing periodontal disease, gingivitis, caries, bad breath, dry mouth, depression, etc. Therefore, the functional substance may have, for example, bactericidal action, bacteriostatic action, biofilm formation inhibition action, anti-inflammatory action, immunomodulatory action, tissue regeneration-promoting action, etc.; and preferably bactericidal action, biofilm formation inhibition action, etc.

In an embodiment, at least one member selected from the group consisting of basic substances (basic functional substances), polyphenols, and lactic acid bacteria may be used as the functional substance. In general, when acidic polysaccharides are mixed with basic functional substances, polyphenols, or lactic acid bacteria, their retention-imparting function is significantly reduced; however, in the present invention, the functional substance is coated with a coating compound, thereby suppressing the reduction in the retention-imparting function.

The basic substance is, for example, a functional substance having an amino group. Basic substances may include peptides such as protamine degradation products, amino acids such as arginine, amino sugars such as glucosamine, amino group-containing polysaccharides such as chitosan, and polyamines such as spermine. Examples of basic substances include protamine degradation products, arginine, lysine, glucosamine, chitosan, spermine, spermidine, putrescine, piperine, organic compounds containing divalent cations (calcium, magnesium, iron, etc.), inorganic compounds containing divalent cations (calcium, magnesium, iron, etc.), and the like. The basic substance is preferably at least one member selected from the group consisting of protamine degradation products, arginine, lysine, glucosamine, and chitosan; more preferably at least one member selected from the group consisting of protamine degradation products, arginine, lysine, glucosamine, and chitosan; and particularly preferably at least one member selected from the group consisting of arginine, lysine, glucosamine, and chitosan.

Protamine is a strongly basic protein that is present as a nucleoprotamine bound to deoxyribonucleic acid in sperm nuclei of fish, such as salmon, herring, and trout. A protamine degradation product can be produced by degrading protamine by hydrolysis, physical cleavage (e.g., sonication), or the like. The protamine degradation product is preferably a hydrolysate obtained by enzymatic hydrolysis of protamine. The details of the protamine degradation product and the method for producing the protamine degradation product are described in PTL 1, and the same applies to the present specification.

Examples of organic compounds containing divalent cations (calcium, magnesium, iron, copper, etc.) include hem iron, calcium pantothenate, calcium gluconate, zinc gluconate, and the like. Examples of organic mixtures include zinc yeast, selenium yeast, manganese yeast, chromium yeast, and the like. These can be used singly or in combination of two or more.

Examples of inorganic compounds containing divalent cations (calcium, magnesium, iron, copper, etc.) include magnesium chloride, calcium hydroxide, magnesium hydroxide, calcium acetate, calcium carbonate, iron oxide, magnesium sulfate, zinc sulfate, calcium silicate, and the like. These can be used singly or in combination of two or more.

The polyphenol may be a substance having two or more hydroxyl groups attached to an aromatic ring. The polyphenol may have antibacterial, bad breath control, and anti-inflammatory effects. The polyphenol may have antibacterial, bad breath control, and anti-inflammatory effects in the oral cavity. Examples of polyphenols include polyphenolic compounds, lignan compounds, propolis, finely ground polyphenol-containing plants, polyphenol-containing extracts prepared by extracting plants with hot water and/or organic solvents (e.g. methanol, ethanol, and acetone), and the like. These can be used singly or in combination of two or more.

Examples of polyphenolic compounds include chlorogenic acid, quercetin, resveratrol, tannic acid, caffeic acid, caffeic acid phenethyl ester, hydroxytyrosol, oleuropein, gallic acid, eugeniin, daidzein, procyanidin, theaflavin, catechins (catechin, epicatechin, epigallocatechin, epicatechin gallate, epigallocatechin gallate, etc.), anthocyanidins (cyanidin, delphinidin, etc.), morin, taxifolin, manniflavanone, and the like. Preferred polyphenolic compounds are chlorogenic acid, quercetin, resveratrol, tannic acid, catechins, anthocyanidins, caffeic acid, gallic acid, eugeniin, procyanidin, theaflavin, and the like; and more preferred are chlorogenic acid, quercetin, resveratrol, tannic acid, catechins, anthocyanidins, and the like. The polyphenolic compounds can be used singly or in combination of two or more.

Examples of lignan compounds include syringin (eleutheroside B), eleutheroside E, dehydrodiconiferyl alcohol, dihydrodehydrodiconiferyl alcohol, syringaresinol, pinoresinol, epoxylyoniresinol, lyoniresinol, lariciresinol, secoisolariciresinol, matairesinol, sesamin, sesaminol, sesamolinol, and the like; and preferably syringin (eleutheroside B) and eleutheroside E. The lignan compounds can be used singly or in combination of two or more.

The polyphenol-containing plant may be a plant containing a polyphenolic compound. Examples of the polyphenol-containing plant include tea (green tea, black tea, Kuding tea, etc.), cassis, coffee beans, guava, blueberries, apples, astringent persimmons, lychees, chestnuts, grapes, cacao, garcinia, Siberian ginseng, and the like. These can be used singly or in combination of two or more.

The polyphenol-containing plant extract may be an extract obtained by extracting the polyphenol-containing plant with hot water, an organic solvent, or the like. Preferred are hot-water extract, methanol extract, ethanol extract, hydrous methanol extract, hydrous ethanol extract, and the like. Examples of the polyphenol-containing plant extract include tea (green tea, black tea, Kuding tea, etc.) extract, cassis extract, green coffee bean extract, guava extract (preferably leaf extract), blueberry extract (preferably leaf extract), olive extract (preferably leaf extract), lychee extract, Canavalia gladiata extract, Sasa veitchii extract, and the like. Preferred are green tea extract, cassis extract, green coffee bean extract, guava extract (preferably leaf extract), blueberry extract (preferably leaf extract), Siberian ginseng extract, etc. The polyphenol-containing plant extracts can be used singly or in combination of two or more.

The polyphenol may be at least one member selected from the group consisting of chlorogenic acid, quercetin, resveratrol, tannic acid, caffeic acid, caffeic acid phenethyl ester, hydroxytyrosol, oleuropein, gallic acid, eugeniin, catechin, daidzein, procyanidin, theaflavin, polyphenol-containing plant extract, propolis, champignon extract, syringin, and eleutheroside E.

The lactic acid bacteria may be live or dead bacteria. The lactic acid bacteria may be in any form, but are preferably in solid or powder form, and more preferably in powder form.

In an embodiment, the functional substance is at least one member selected from the group consisting of protamine degradation products, arginine, lysine, glucosamine, chitosan, chlorogenic acid, quercetin, resveratrol, tannic acid, catechins, anthocyanidins, caffeic acid, gallic acid, eugeniin, procyanidin, theaflavin, syringin, eleutheroside E, lactic acid bacteria, tea extract, cassis extract, green coffee bean extract, guava extract (preferably leaf extract), blueberry extract (preferably leaf extract), olive extract (preferably leaf extract), lychee extract, Canavalia gladiata extract, and Sasa veitchii extract.

In another embodiment, the functional substance is at least one member selected from the group consisting of protamine degradation products, arginine, lysine, glucosamine, chitosan, chlorogenic acid, quercetin, resveratrol, tannic acid, catechins, anthocyanidins, syringin, eleutheroside E, lactic acid bacteria, green tea extract, cassis extract, green coffee bean extract, guava extract (preferably leaf extract), and blueberry extract (preferably leaf extract).

The content of the functional substance in the oral retention composition is, for example, 0.1 mass% to 80 mass% or 0.1 mass% to 50 mass% based on the total mass of the composition, preferably 1 mass% to 40 mass%, and more preferably 1 mass% to 20 mass%. The content of the functional substance within this range enhances the retention of the functional component in the oral cavity.

### Coating Compound

The coating compound is at least one compound selected from the group consisting of shellac and zein. The functional substance is coated with the coating compound to form a coated functional substance, whereby the functional substance is less likely to impair the retention-imparting function of the acidic polysaccharide. In the present invention, it is sufficient that the coating compound coats the functional substance, such as a protamine degradation product. In addition, components constituting the oral retention composition, other than the functional substance, may also be coated with the coating compound.

In the present specification, coating refers to a state of an object to be coated (e.g., a functional substance) whose surface is partially or entirely coated with a film (e.g., a film containing a coating compound etc.). Further, coating also includes a mode in which the object to be coated is contained in the film, as in the case of coating the acidic polysaccharide or other constituents using a liquid containing the object to be coated and the coating compound. For example, even if part or all of the acidic polysaccharide is coated with ethanol or hydrous ethanol containing a protamine degradation product and shellac, when the surface of the protamine degradation product is partially or entirely coated with shellac, it corresponds to the shellac-coated protamine degradation product.

The degree of coating may be a degree that can be achieved by a general granulation method, and it is not necessary that 100% of the surface of the functional substance is coated with the coating compound. The degree of coating is, for example, 50% or more, 60% or more, 70% or more, 80% or more, 85% or more, 90% or more, or 95% or more, preferably 97% or more, more preferably 98% or more, even more preferably 99% or more, and particularly preferably 100%. The degree of coating can be determined by image analysis using X-ray CT. The details of the determination method are as follows. Since X-ray CT measurement can visualize the surface and inside of a substance and output them as 3D images, the surface state of the substance after coating can be clarified by measuring the substance before and after coating. First, the substance before coating is measured by X-ray CT, and a 3D image is output. Next, a 3D image of the coated substance is similarly output. Both images are compared, and the area of the different portion, i.e., the coated portion, is determined by software calculation. This area is divided by the total area of the substance to determine the coverage.

The coating compound is preferably shellac in terms of high drying efficiency. Shellac is used as a brightening agent, a masking agent, a coating agent, or the like in the field of food, quasi-drug, etc. These can also be used in the present invention.

Zein is used as a coating agent in the field of food, drug, etc. These can also be used in the present invention.

The content of the coating compound in the oral retention composition is, for example, 0.001 mass% to 40 mass%, 0.001 mass% to 25 mass%, 0.01 mass% to 20 mass%, or 0.01 mass% to 15 mass%, preferably 0.001 mass% to 10 mass%, and more preferably 0.01 mass% to 8 mass%, based on the total mass of the composition. The content of the coating compound within this range enhances the retention of the functional component in the oral cavity. Further, the content of the coating compound can be, for example, 0.1 to 50 parts by mass or 1 to 40 parts by mass, based on 100 parts by mass of the functional substance, preferably 1 to 30 parts by mass, and more preferably 1 to 20 parts by mass.

### Acidic Polysaccharide

The acidic polysaccharide has the function of imparting oral retention to the functional substance. The acidic polysaccharide is mixed with the coated functional substance to form an oral retention composition. Mixing of the acidic polysaccharide with the coated functional substance makes it possible to suppress the adverse effects of the functional substance on the function of imparting oral retention (e.g., viscosity) of the acidic polysaccharide.

Examples of the acidic polysaccharide include carrageenan, hyaluronic acid, xanthan gum, sodium alginate, pectin, gum arabic, and the like. These can be used singly or in combination of two or more. Preferred acidic polysaccharides are carrageenan, hyaluronic acid, xanthan gum, sodium alginate, and the like; and more preferred acidic polysaccharides are carrageenan, hyaluronic acid, gum arabic, sodium alginate, and the like. Particularly preferred among these are carrageenan and sodium alginate, because they can increase the oral retention time of the functional substance.

The content of the acidic polysaccharide in the oral retention composition is, for example, 0.1 mass% to 90 mass%, 1 mass% to 90 mass%, 0.1 mass% to 60 mass%, or 1 mass% to 60 mass%, based on the total mass of the composition, and preferably 1 mass% to 30 mass%. The content of the acidic polysaccharide within this range is preferable because the oral retention time of the functional substance can be increased.

The oral retention composition may also contain an excipient in addition to the coated functional substance and acidic polysaccharide. The oral retention composition, when in the form of granules, tablets, or a finely ground product, preferably contains an excipient because the formability increases.

The average particle size (D50) of the oral retention composition can be, for example, 1 µm to 5000 µm or 1 µm to 400 µm, preferably 10 µm to 1000 µm, more preferably 50 µm to 400 µm, and particularly preferably 1 µm to 400 µm. The average particle size is determined by the method described in the “Measurement Method for Particle Size” section in the Examples.

The excipient may be selected from those available in the field of food, drug, quasi-drug, etc., according to the shape, application, granulation method, etc. The amount of excipient used may be an amount necessary to form a desired shape etc., and is, for example, 90 parts by mass or less, 80 parts by mass or less, 70 parts by mass or less, 0.001 to 90 parts by mass, 0.001 to 80 parts by mass, 0.001 to 70 parts by mass, 0.01 to 90 parts by mass, 0.01 to 80 parts by mass, or 0.01 to 70 parts by mass, per part by mass of the total amount of the functional substance, coating compound, and acidic polysaccharide.

The oral retention composition is preferably in the form of granules, powder, or tablets, and more preferably granules or powder. Products in these forms to be taken or ingested as granular tablets, powder, tablets, orally disintegrating tablets, foaming tablets, capsules, or the like are defined as oral retention compositions. Products for oral cavity and oral products containing oral retention compositions in these forms are respectively defined as compositions for oral cavity and oral compositions. For example, compositions for oral cavity and oral compositions are generally viable products without containing oral retention compositions; however, oral retention compositions may be added thereto in expectation of the function and retention of the oral retention compositions in the oral cavity.

The oral retention composition can be used as a material for producing a composition for oral cavity or an oral composition. The composition for oral cavity or oral composition can be produced by applying a method generally used in the production of compositions for oral cavity or oral compositions to the oral retention composition and other materials used for compositions for oral cavity or oral compositions. For example, an oral retention composition in granular form can be used in the production of chewing gums, candies, gummies, and the like that contain the oral retention composition in granular form.

### Composition for Oral Cavity

Examples of the composition for oral cavity include granules, toothpaste, oil-based ointments, oral care products such as films, drugs (lozenges etc.), quasi-drugs (lozenges etc.), chewable tablets, and other compositions applicable to the oral cavity and not included in food. Preferred among these are granules, lozenges, and chewable tablets.

Applications of the composition for oral cavity include bad breath control, taste retention, taste masking, and the like. Applications for bad breath control include bad breath control while wearing a mask, bad breath control at the time of awakening, and the like.

The content of the oral retention composition in the composition for oral cavity may be suitably selected depending on the product form of the composition for oral cavity, the mode of use, the final content concentration of the functional substance, etc. Although it is not necessarily appropriate to define the range of content uniformly, the content is, for example, 0.1 mass% to 50 mass%, 0.1 mass% to 40 mass%, 1 mass% to 50 mass%, 1 mass% to 40 mass%, or 0.1 mass% to 30 mass%, preferably 1 mass% to 30 mass%, and more preferably 1 mass% to 20 mass%.

The composition for oral cavity can be obtained by adding the oral retention composition to a known composition for oral cavity, or by replacing part or all of the components that constitute a known composition for oral cavity with the oral retention composition. Although not particularly limited, the composition for oral cavity can be produced by appropriately changing the method for producing a known composition for oral cavity.

### Oral Composition

Examples of the oral composition include orally ingested compositions (mainly food), such as chewing gums, hard candies (lollipops), soft candies, gummies, jellies, chewable tablets, and tablet confectionery such as ramune (porous and chalky soft candy). Preferred are food that is not swallowed immediately but is kept in the oral cavity for a certain period of time, and food that is expected to attach functional substances to teeth or gums when chewed, such as chewing gums, hard candies, soft candies, and gummies.

Applications of the oral composition include bad breath control, taste retention, taste masking, and the like. Applications for bad breath control include bad breath control while wearing a mask, bad breath control at the time of awakening, and the like.

The content of the oral retention composition in the oral composition may be suitably selected depending on the product form of the oral composition, the mode of use, the final content concentration of the functional substance, etc. Although it is not necessarily appropriate to define the range of content uniformly, the content is, for example, 0.1 mass% to 50 mass%, 0.1 mass% to 40 mass%, 1 mass% to 50 mass%, 1 mass% to 40 mass%, 0.1 mass% to 30 mass%, or 0.5 mass% to 20 mass%, preferably 1 mass% to 30 mass%, and more preferably 1 mass% to 20 mass%.

The oral composition can be obtained by adding the oral retention composition to a known oral composition, or by replacing part or all of the components that constitute a known oral composition with the oral retention composition. Although not particularly limited, the oral composition can be produced by appropriately changing the method for producing a known oral composition (particularly food).

### Method for Producing Oral Retention Composition

The method for producing an oral retention composition comprising at least one compound selected from the group consisting of shellac and zein, a functional substance, and an acidic polysaccharide, the functional substance coated with the at least one compound selected from the group consisting of shellac and zein being mixed with the acidic polysaccharide;

the method comprising:
step 1 of mixing the at least one compound selected from the group consisting of shellac and zein dissolved or dispersed in a solvent with the functional substance so that the compound coats the functional substance; and
step 2 of mixing the functional substance coated with the at least one compound selected from the group consisting of shellac and zein obtained in step 1 with the acidic polysaccharide.

In step 1, a functional substance is coated with a coating compound. For example, the functional substance is mixed with a solution of the coating compound dissolved or dispersed in a solvent, and optionally the liquid component is removed after mixing, thereby obtaining a coated functional substance.

The amount of the coating compound used is, for example, 0.1 to 50 parts by mass or 1 to 40 parts by mass based on 100 parts by mass of the functional substance, preferably 1 to 30 parts by mass, and more preferably 1 to 20 parts by mass.

As the solvent, water, ethanol, or hydrous ethanol can be used, and ethanol and hydrous ethanol are preferred. As the hydrous ethanol, for example, hydrous ethanol with an ethanol concentration of 60% to 99% (v/v), preferably 60% to 95% (v/v), and more preferably 80% to 90% (v/v), can be used. The amount of solvent used is, for example, 5 parts by mass to 40 parts by mass, and preferably 10 parts by mass to 30 parts by mass, per part by mass of the coating compound.

When the coating compound is shellac, the solvent is preferably ethanol because shellac is easily dissolved. When the coating compound is zein, the solvent is preferably hydrous ethanol with an ethanol concentration of 60% to 99% (v/v), and more preferably hydrous ethanol with an ethanol concentration of 80% to 90% (v/v), because zein is easily dissolved.

The mixing method is not particularly limited as long as the coating compound can coat the surface of the functional substance. Examples include dry granulation, wet granulation, and the like, and wet granulation is preferred. After mixing, the liquid component may be removed. Examples of the removal method include hot-air drying (25° C. to 100° C.), vacuum drying, and the like.

In step 2, the functional substance coated with at least one compound selected from the group consisting of shellac and zein obtained in step 1 is mixed with an acidic polysaccharide. At this time, an excipient may be mixed, if necessary.

The amount of acidic polysaccharide used is, for example, 0.1 parts by mass to 50 parts by mass, and preferably 1 part by mass to 30 parts by mass, per part by mass of the coated functional substance.

Depending on the form of the oral retention composition, mixing may be performed by a known method applicable to that form. For example, the coated functional substance and a powdery acidic polysaccharide are mixed and stirred, and then alcohol such as ethanol, or hydrous alcohol such as hydrous ethanol is added, followed by granulation. It is preferable, in terms of kneading efficiency, to mix the functional substance and the acidic polysaccharide while adding (preferably dropping) hydrous alcohol.

The alcohol is, for example, ethanol or methanol, and preferably ethanol. The hydrous alcohol is, for example, hydrous ethanol or hydrous methanol, and preferably hydrous ethanol. The alcohol concentration of the hydrous alcohol is, for example, 50% to 99% or 50% to 95% (v/v), preferably 70% to 95% (v/v), and more preferably 80% to 90% (v/v).

### EXAMPLES

The following describes an embodiment of the present invention in more detail with references to Examples. However, the present invention is not limited to the Examples. The unit “mass%” in the Examples indicates “% (w/w)” unless otherwise specified. In the Examples, the materials listed below were used.
Carrageenan: SATIAGUM MM 30; Unitec Foods Co., Ltd.
Protamine degradation product: HAP100; Maruha Nichiro Corporation. This is a hydrolysate of protamine hydrochloride derived from the milt of chum salmon (shiro sake) (trade name: protamine, Maruha Nichiro Corporation) due to bromelain (Amano Enzyme Inc.) and contains four types of basic peptides.
Arginine: L-arginine; Ajinomoto Co., Inc.
Glucosamine: MSM60; Bio Actives Japan Corporation
Chitosan: KOYO Chitosan FM-80; Koyo Chemical Co., Ltd.
N-acetylglucosamine: Bio-NAG; BHN Co., Ltd.
Propolis: propolis extract powder; Nihon Yoho Co., Ltd.
Royal jelly: royal jelly FD powder; Nakahara Co., Ltd.
Green tea extract (green tea extract powder): Teafuran 30A; Ito En, Ltd., Sunphenon 90S; Taiyo Kagaku Co., Ltd.
Cassis extract (cassis extract powder): active cassis extract 35; Tama Biochemical Co., Ltd.
Coffee bean extract (green coffee bean extract powder): Phytopharma Co., Ltd.
Blueberry extract (blueberry leaf extract powder): Bizen Chemical Co., Ltd.
Guava extract (guava leaf extract powder): Bizen Chemical Co., Ltd.
Chlorogenic acid: 08416-14; Nacalai Tesque, Inc.
Tannic acid: 40001-32; Kanto Chemical Co., Inc.
Quercetin: P0042; Tokyo Chemical Industry Co., Ltd.
Resveratrol: Fujifilm Wako Pure Chemical Corporation
Shellac: Gifu Shellac Manufacturing Co., Ltd.
Zein (decomposed corn): Kobayashi ZEIN DP-N; Kobayashi Perfumery Co., Ltd.
HPMC (hydroxypropyl methylcellulose): Metolose SE-06; Shin-Etsu Kasei Kogyo Co., Ltd.
Hyaluronic acid: Hyaluronic acid RV; Nippon Shinyaku Co., Ltd.
Xanthan gum: Bistop; San-Ei Gen F.F.I., Inc.
Sodium alginate: sodium alginate I-1; Kimica Corporation
Crystalline cellulose: FD-F20; Asahi Kasei Corporation, VIVAPUR101;
Sucralose: Sansweet 200; San-Ei Gen F.F.I., Inc.
Aspartame: Pal Sweet; Ajinomoto Co., Inc.
Xylitol: Xylit; Mitsubishi Shoji Foodtech Co., Ltd.
Lactitol: Lactitol MC; Mitsubishi Shoji Foodtech Co., Ltd.
Siberian ginseng extract powder: UKOGIN; Bizen Chemical Co., Ltd.
Epigallocatechin gallate standard preparation; Fujifilm Wako Pure Chemical Corporation
Syringin: purity 95% or higher; Cayman Chemical Company
Eleutheroside E: purity 98% or higher; Sigma-Aldrich
Spermidine: purity 95% or higher; Fujifilm Wako Pure Chemical Corporation
Fine silicon dioxide: Sylopage 720; Fuji Silysia Chemical, Ltd.
Calcium stearate: Eika Corporation
Lactic acid bacteria powder: L8020 lactic acid bacteria powder; Bizen Chemical Co., Ltd.

### Test Example 1: Change in Viscosity of Acidic Polysaccharide Due to Functional Substance

The viscosity of acidic polysaccharides is important for their oral retention. The viscosity of an acidic polysaccharide was measured and evaluated as described below. Measurement device: a plastic plate (18 cm × 32 cm) was inclined at 10° to the horizontal plane using a protractor to create an inclined plane.

Measurement: 300 µL of a sample was dropped at a predetermined point on the inclined plane; after 3 minutes, the distance over which the sample traveled was measured (progress distance). Sample: a control sample that is an aqueous solution containing 1 mass% of an acidic polysaccharide, an uncoated sample that is an aqueous solution containing a mixture of a predetermined amount of an uncoated functional substance and an acidic polysaccharide (acidic polysaccharide: 1 mass%), and a coated sample that is an aqueous solution containing a mixture of a predetermined amount of a functional substance coated with a coating compound and an acidic polysaccharide (acidic polysaccharide: 1 mass%). Evaluation method: the relative degree of progress (RD) was calculated from the following formula. The progress distance (mm) of the control sample is D0, the progress distance (mm) of the uncoated sample is D1, and the progress distance (mm) of the coated sample is D2.

$$\text{Relative degree of progress} \left(\text{RD}\right) = \left(\text{D2} \text{−} \text{D0}\right) / \left(\text{D1} \text{−} \text{D0}\right)$$

RD < 1.0 indicates that the decrease in viscosity of an aqueous solution of an acidic polysaccharide caused by a functional substance has been limited by coating. RD > 1.0 indicates that the decrease in viscosity of an aqueous solution of an acidic polysaccharide caused by a functional substance has been promoted. RD = 1.0 indicates that the viscosity of an aqueous solution of an acidic polysaccharide does not change regardless of the presence or absence of coating. An RD value greater than 0.9 was evaluated as “-” because the decrease in viscosity was not limited or was limited only slightly, while an RD value of 0.9 or lower was evaluated as “A” because the decrease in viscosity was limited.

The control sample, uncoated sample, and coated sample were prepared as described below, and used in evaluation of the viscosity of aqueous carrageenan solutions. Table 1 shows the results.

An acidic polysaccharide was dissolved in water to prepare a 1 mass% aqueous solution, which was determined to be a control sample. Separately, the same solution as the control sample was prepared, and 0.75 mass% of a functional substance (if the functional substance is a protamine degradation product, 0.68 mass%) was added and mixed. The obtained solution was determined to be an uncoated sample. Separately, ethanol in which shellac was dissolved at a concentration of 25 mass% was dropped onto a powdery functional substance. The ethanol was added in such an amount that the amount of shellac was 10 mass% with respective to the mass of the functional substance. After they were sufficiently mixed, the mixture was dried in a dryer at 50° C., thereby preparing a granular coated functional substance. A coated functional substance in such an amount as to give a functional substance concentration of 0.75 mass% (if the functional substance is a protamine degradation product, 0.68 mass%) was mixed with 20 g of the same solution as the control solution (an aqueous solution of 1 mass% acidic polysaccharide), and the obtained solution was determined to be a coated sample.

TABLE 1

| | Functional Substance | Coating Compound | Acidic Polysaccharide | D1-D0 (mm) | D2-D0 (mm) | RD (Evaluation) |
|---|---|---|---|---|---|---|
| Example 1 | Protamine Degradation Product | Shellac | Carrageenan | 17 | 4 | 0.2 (A) |
| Example 2 | Arginine | Shellac | Carrageenan | 18 | 4 | 0.2 (A) |
| Example 3 | Glucosamine | Shellac | Carrageenan | 16 | 3 | 0.2 (A) |
| Example 4 | Chitosan | Shellac | Carrageenan | 19 | 3 | 0.2 (A) |
| Example 5 | Green Tea Extract | Shellac | Carrageenan | 28 | 6 | 0.2 (A) |
| Example 6 | Cassis Extract | Shellac | Carrageenan | 35 | 7 | 0.2 (A) |
| Example 7 | Coffee Bean Extract | Shellac | Carrageenan | 25 | 5 | 0.2 (A) |
| Example 8 | Guava Extract | Shellac | Carrageenan | 16 | 3 | 0.2 (A) |
| Example 9 | Blueberry Extract | Shellac | Carrageenan | 16 | 2 | 0.1 (A) |
| Example 10 | Propolis | Shellac | Carrageenan | 28 | 5 | 0.2 (A) |
| Comparative Example 1 | N-acetylglucosamine | Shellac | Carrageenan | 1 | 1 | 1 (-) |
| Comparative Example 2 | Royal Jelly | Shellac | Carrageenan | 1 | 1 | 1 (-) |

### Protamine Degradation Product

Carrageenan showed a significant decrease in viscosity when mixed with an uncoated protamine degradation product (D1-D0 in Example 1 is 17 mm); the sample went down the slope to a greater degree as compared with carrageenan with its original viscosity. PTL 1 suggested that carrageenan was reacted with a protamine degradation product and adsorbed the protamine degradation product, and that xanthan gum, which is also an acidic polysaccharide just as carrageenan, was also reacted with a protamine degradation product and adsorbed the protamine degradation product, resulting in a decrease in antimicrobial activity of the protamine degradation product. However, PTL 1 nowhere indicated that a change in viscosity of acidic polysaccharides occurs; this was a new finding.

Evaluation of a protamine degradation product coated with shellac found a decrease in the change in viscosity (D2-D0 in Example 1 is 4 mm), and its viscosity was close to the original viscosity of carrageenan. RD was lower than 0.9, which was probably because shellac coating was able to inhibit the reaction and adsorption between the protamine degradation product and carrageenan. PTL 1 confirmed antimicrobial activity by using a neutral polysaccharide with a low adsorption rate; thus, it was thought that the inhibition of the reaction and adsorption by shellac coating would sufficiently prevent an acidic polysaccharide from inhibiting antimicrobial activity of a protamine degradation product, and would also sufficiently limit the decrease in viscosity of the acidic polysaccharide.

### Basic Functional Substance

Because arginine, glucosamine, and chitosan, which are basic substances, also decreased the viscosity of carrageenan (D1-D0 in Examples 2 to 4 is 16 to 19 mm), these were thought to have reacted with carrageenan in the same manner as the protamine degradation product. However, N-acetylglucosamine, which is a glucosamine derivative, (neutral compound) did not cause a change in viscosity of carrageenan (D1-D0 in Comparative Example 1 is 1 mm). These results clearly indicate that basic compounds react with carrageenan to decrease the viscosity.

Samples containing shellac-coated arginine, glucosamine, or chitosan also showed a limited decrease in viscosity as in Example 1, and the viscosity was close to the original physical properties of carrageenan (D2-D0 in Examples 2 to 4 is 3 or 4 mm). RD was lower than 0.9, and shellac coating was able to inhibit the reaction and adsorption between the basic functional substances and carrageenan. It was thought that the inhibition of the reaction and adsorption due to shellac coating would sufficiently prevent an acidic polysaccharide from inhibiting biofilm formation inhibition activity of a functional substance, and would also sufficiently limit a decrease in viscosity of the acidic polysaccharide. However, RD of N-acetylglucosamine was 1, and N-acetylglucosamine did not show reactivity with carrageenan.

### Plant Extract, Propolis, and Royal Jelly

Because a green tea extract, a cassis extract, a coffee bean extract, a guava extract, a blueberry extract, and propolis decreased the viscosity of carrageenan in the same manner as the basic functional substances (D1-D0 in Examples 5 to 10 is 16 to 35 mm), these extracts were thought to have reacted with carrageenan in the same manner as the protamine degradation product. However, RD of royal jelly was 1, and there was little difference between D1 and D0; thus, royal jelly was not considered to be reactive with carrageenan.

Samples containing a shellac-coated plant extract or propolis showed a limited decrease in viscosity as in Example 1, and the viscosity was close to the original physical properties of carrageenan (D2-D0 in Examples 5 to 10 is 2 to 7 mm). RD was lower than 0.9, and shellac coating was able to inhibit the reaction and adsorption between the plant extract or propolis and carrageenan. It was thought that the inhibition of the reaction and adsorption due to shellac coating would sufficiently prevent an acidic polysaccharide from inhibiting antimicrobial activity of the functional substances, and would also sufficiently limit a decrease in viscosity of the acidic polysaccharide. However, RD of N-acetylglucosamine was 1, and N-acetylglucosamine did not show reactivity with carrageenan.

Plant extracts generally contain polyphenols, and the five extracts used in this study are known to be rich in catechins, anthocyanidins, chlorogenic acid, tannic acid, etc. Propolis is also known to contain high levels of flavonoids, which are also polyphenols. However, royal jelly, which is a honey bee secretion like propolis, contains no flavonoids. It is the first finding that a substance with a relatively high polyphenol content, such as plant extracts and propolis, reacts with carrageenan and decreases the viscosity of carrageenan. It appears that polyphenolic compounds react with carrageenan to decrease the viscosity.

### Test Example 2: Change in Viscosity of Acidic Polysaccharide Due to Polyphenolic Compound

Chlorogenic acid, tannic acid, quercetin, and resveratrol were prepared as polyphenolic compounds. The effects of these substances on the viscosity of carrageenan were measured and their reactivity was evaluated.

The measurement device, measurement, control sample, and D0 were the same as those in Test Example 1. An aqueous methanol solution containing a polyphenolic compound was prepared by dissolving the polyphenolic compound in an aqueous methanol solution (methanol concentration: 50 mass%) and diluted to 1 mg/mL in a measuring cylinder. 200 µL of this solution was added to 20 g of the same solution as the control sample (aqueous solution of 1 mass% carrageenan) and homogeneously mixed. The obtained solution was determined to be an uncoated sample. Additionally, a solution was prepared in the same manner as the uncoated sample, except that a 50% (v/v) aqueous methanol solution was used instead of the aqueous methanol solution containing a polyphenolic compound and determined to be a methanol sample. Evaluation Method: The progress distance (mm) of the control sample is D0. The progress distance (mm) of the uncoated sample is D1. The progress distance (mm) of the methanol sample is D1M. The reactivity index (RI) between a polyphenolic compound and carrageenan was calculated according to the following formula.

$$\text{Reactivity Index} \left(\text{RI}\right) = \left(\text{D1} \text{−} \text{D0}\right) / \left(\text{D1M} \text{−} \text{D0}\right)$$

In this test system, the addition of an aqueous solution causes some decrease in viscosity due to a dilution effect. Thus, the determination method described above was used to detect the changes in viscosity due to the effect of a polyphenolic compound. Note that ±10% of the RI value was assumed to be within the margin of error (i.e., an RI value of 0.9 to 1.1 was assumed to be within the margin of error).

1.1<RI indicates that the viscosity of carrageenan was decreased by a polyphenolic compound. 0.9≤RI≤1.1 indicates that the viscosity of carrageenan was not changed by a polyphenolic compound. RI<0.9 indicates that the viscosity of carrageenan was increased by a polyphenolic compound. Table 2 shows the results.

TABLE 2

| | | RI |
|---|---|---|
| Comparative Example 3 | Chlorogenic Acid | 4.1 |
| Comparative Example 4 | Tannic Acid | 2.5 |
| Comparative Example 5 | Quercetin | 2.2 |
| Comparative Example 6 | Resveratrol | 3.5 |

The polyphenolic compound solutions (uncoated samples) exhibited a decrease in viscosity greater than the solution prepared by adding a 50% aqueous methanol solution (methanol sample). Thus, the calculated RI was 2.5 to 4.1. Given the fact that these polyphenolic compounds decreased the viscosity of aqueous carrageenan solutions when added in an amount of only 10 ppm, and that chlorogenic acid, tannic acid, quercetin, resveratrol, and their derivatives are abundant in plant extracts, plant extracts containing polyphenols (not only the plant extracts confirmed to be reactive with carrageenan in Test Example 1, but also other plant extracts) would react with carrageenan and decrease the viscosity.

### Test Example 3: Change in Viscosity of Acidic Polysaccharide Due to Coated Polyphenolic Compound

A test for reaction inhibition effects due to shellac coating was performed by using chlorogenic acid, tannic acid, quercetin, and resveratrol, which were shown to be reactive with carrageenan and decrease its viscosity in Test Example 2.

The compounds were each dissolved in a 50% aqueous methanol solution, and diluted to 10 mg/mL concentration in a measuring cylinder. 50 g of a 10 mass% aqueous dextrin solution (Pinedex #100, Matsutani Chemical Industry Co., Ltd.) was added to 20 mL of each of the obtained aqueous solutions, and homogeneously mixed, followed by performing a freeze-drying treatment, thereby preparing individual powder of each compound (coated functional substance). Individual powder was mixed with 20 g of a 1 mass% aqueous carrageenan solution, thereby obtaining uncoated samples. Separately, the same solutions as the uncoated samples and a solution of a 25 mass% coating compound in ethanol were prepared. The prepared ethanol solution was added dropwise to the prepared, same solutions as the uncoated samples such that the amount of shellac was 10 mass% of the individual functional substance, and sufficiently mixed, followed by drying in a dryer at 50° C., thereby preparing individual granular coated functional substances. The coated functional substances each in such an amount as to give a functional substance concentration of 0.75 mass% were mixed with 20 g of the same solution as the control sample (aqueous solution of 1 mass% acidic polysaccharide) to prepare individual solutions, and these solutions were determined to be coated samples.

D0, D1, and D2 of the obtained samples were measured in the same manner as in Test Example 1, and their RD values were calculated. Table 3 shows the results.

TABLE 3

| | Functional Substance | Coating Compound | Acidic Polysaccharide | D1-D0 (mm) | D2-D0 (mm) | RD (Evaluation) |
|---|---|---|---|---|---|---|
| Example 11 | Chlorogenic Acid | Shellac | Carrageenan | 34 | 7 | 0.2 (A) |
| Example 12 | Tannic Acid | Shellac | Carrageenan | 30 | 5 | 0.2 (A) |
| Example 13 | Quercetin | Shellac | Carrageenan | 25 | 4 | 0.2 (A) |
| Example 14 | Resveratrol | Shellac | Carrageenan | 32 | 5 | 0.2 (A) |

Although the polyphenolic compounds reacted with carrageenan and decreased the viscosity of the solutions (D1-D0: 25 to 34 mm), shellac coating limited the decrease in viscosity with an RD of lower than 0.9, indicating the inhibition of their reaction with carrageenan. These results are similar to those obtained with the plant extracts in Examples 5 to 10, suggesting that the coating of the plant extracts inhibited the action of the polyphenolic compounds in the plant extracts on carrageenan and thereby inhibited their reaction with carrageenan.

From the above results, it was assumed that the action of inhibiting the decrease in viscosity of acidic polysaccharides by a coating compound would be achieved not only in the test examples above, but also in basic functional substances, polyphenolic compounds, plant extracts containing polyphenolic compounds, secretions containing flavonoids such as propolis, or substances containing these.

### Test Example 4: Concentration and Type of Coating Compound

D0, D1, D2, and RD were determined in the same manner as in Test Example 1 except that the shellac concentration based on the mass of the functional substance was changed. The shellac concentration was 1, 5, 10, and 20 mass% of the mass of the protamine degradation product. Additionally, D0, D1, D2, and RD were determined in the same manner as in Test Example 1, except that the coating compound was zein or HPMC, and the solution of a 25 mass% coating compound in ethanol was replaced with hydrous ethanol with an ethanol concentration of 80% (v/v) in which zein or HPMC was dissolved at 25 mass%. For the functional substance, a protamine degradation product was used in an amount of 0.68 mass% as in Test Example 1. Table 4 shows the results.

TABLE 4

| | Functional Substance | Coating Compound | Acidic Polysaccharide | D1-D0 (mm) | D2-D0 (mm) | RD (Evaluation) |
|---|---|---|---|---|---|---|
| Example 15 | Protamine Degradation Product | Shellac (1%) | Carrageenan | 19 | 11 | 0.6 (A) |
| Example 16 | Protamine Degradation Product | Shellac (5%) | Carrageenan | 19 | 5 | 0.3 (A) |
| Example 17 | Protamine Degradation Product | Shellac (10%) | Carrageenan | 19 | 2 | 0.1 (A) |
| Example 18 | Protamine Degradation Product | Shellac (20%) | Carrageenan | 19 | 2 | 0.1 (A) |
| Example 19 | Protamine Degradation Product | Zein (10%) | Carrageenan | 16 | 8 | 0.5 (A) |
| Comparative Example 7 | Protamine Degradation Product | HPMC (10%) | Carrageenan | 16 | 22 | 1.4 (A) |

Even shellac coating in an amount of 1 mass% of a protamine degradation product limited the decrease in viscosity of the aqueous carrageenan solution containing the protamine degradation product (RD in Example 15 was 0.6). The decrease in viscosity was limited dependently on the amount of shellac (Examples 15 to 18). Because all of Examples 15 to 18 had an RD of lower than 0.9, shellac within the range of at least 1 to 20 mass% of the functional substance was found to be effective in inhibiting the reaction with carrageenan.

Coating with zein resulted in an RD of lower than 0.9 (Example 19), confirming the inhibition of the reaction between the protamine degradation product and carrageenan. In contrast, coating with HPMC promoted the decrease in viscosity of carrageenan, resulting in an RD greater than 1.1 (Comparative Example 7). HPMC coating appeared to decrease the oral retention due to carrageenan.

### Test Example 5: Type of Acidic Polysaccharides

Carrageenan was changed to other acidic polysaccharides. The effect of shellac coating on the viscosity of the acidic polysaccharides was measured. The procedure of Test Example 1 was repeated, except that carrageenan was replaced with hyaluronic acid, xanthan gum, or sodium alginate. Table 5 shows the results.

TABLE 5

| Functional Substance | Coating Compound | Acidic Polysaccharide | D1-D0 (mm) | D2-D0 (mm) | RD (Evaluation) |
|---|---|---|---|---|---|
| Example 20 Protamine Degradation Product | Shellac (10%) | Hyaluronic Acid | 16 | 8 | 0.5 (A) |
| Example 21 Protamine Degradation Product | Shellac (10%) | Xanthan Gum | 43 | 17 | 0.3 (A) |
| Example 22 Protamine Degradation Product | Shellac (10%) | Sodium Alginate | 14 | 5 | 0.4 (A) |

Hyaluronic acid, xanthan gum, and sodium alginate (acidic polysaccharides) reacted with the protamine degradation product, resulting in a decreased viscosity of their 1% aqueous solution. Although the decrease in viscosity of hyaluronic acid or sodium alginate (D1-D0 in Examples 20 and 22 was 16 and 14 mm) was similar to that of carrageenan (D1-D0 in Example 1 was 17 mm), the viscosity of xanthan gum in particular was greatly decreased (D1-D0 in Example 21 was 43 mm). However, shellac coating substantially limited the decrease in viscosity in the same manner as the case of carrageenan (RD in Examples 20 to 22 was 0.3 to 0.5). Shellac coating was considered to be effective in limiting the decrease in viscosity of acidic polysaccharides other than carrageenan and thereby limiting the decrease in oral retention.

### Test Example 6: Amount of Acidic Polysaccharide

Granular formulations were prepared according to the formulas shown in Table 6. The unit for the values in Table 6 is mass%.

TABLE 6

| | Comparative Example 8 | Example 23 | Comparative Example 9 | Example 24 | Comparative Example 10 | Example 25 |
|---|---|---|---|---|---|---|
| Protamine Degradation Product | 10 | 10 | 10 | 10 | 10 | 10 |
| Shellac | 0 | 1 | 0 | 1 | 0 | 1 |
| Carrageenan | 1 | 1 | 20 | 20 | 89 | 89 |
| Crystalline Cellulose | 89 | 88 | 70 | 69 | 1 | 0 |
| Total | 100 | 100 | 100 | 100 | 100 | 100 |

A solution of 25 mass% shellac in ethanol was added dropwise to a protamine degradation product. The amount of shellac added was 10 mass% of the protamine degradation product. After being fully mixed, the mixture was dried in a dryer at 50° C. and classified with a No. 60 sieve (mesh size: 250 µm) to obtain granules that passed through the sieve (coated granules). The coated granules, carrageenan, and crystalline cellulose were mixed, while hydrous ethanol with an ethanol concentration of 90% (v/v) was added dropwise. The mixture was dried in a dryer at 50° C. and classified with a No. 60 sieve (mesh size: 250 µm), followed by evaluating the granules that passed through the sieve. The same procedure was repeated except that the coated granules were replaced with a protamine degradation product, thereby preparing granules containing an uncoated protamine degradation product (uncoated granules).

A solution prepared by mixing 1 g of coated granules or uncoated granules with 20 g of water was determined to be a sample, and the progress distance was measured in the same manner as in Test Example 1. The coated granules of Examples 23, 24, and 25 had a shorter progress distance (D1) than that of the uncoated granules of Comparative Examples 8, 9, and 10. This indicated that coating a functional substance with shellac can limit the decrease in viscosity. The carrageenan content in Comparative Example 8 and Example 23 was as small as 1 mass%; however, since crystalline cellulose is insoluble in water, the viscosity that develops with the addition of water can be evaluated as the viscosity due to carrageenan.

### Test Example 7: Particle Size of Granular Formulation

Granular formulations were prepared according to the formulas with a high concentration of a protamine degradation product shown in Table 7 in the same manner as in Test Example 6. The unit for the values in Table 7 is mass%. However, the sieves for use were No. 30 (mesh size: 500 µm) and No. 60 (mesh size: 250 µm). Granules that passed through the No. 30 sieve but not the No. 60 sieve (Example 26) and granules that passed through the No. 60 sieve (Example 27) were measured for the progress distance in the same manner as in Test Example 1. Granules of Examples 26 and 27 were also subjected to precise particle size measurement. Table 8 shows the results of measuring the particle size.

TABLE 7

| | Examples 26 and 27 | Comparative Example 11 |
|---|---|---|
| Protamine Degradation Product | 40 | 40 |
| Shellac | 4 | 0 |
| Carrageenan | 10 | 10 |
| Crystalline Cellulose | 46 | 50 |
| Total | 100 | 100 |

### Measurement Method for Particle Size

The particle size was measured with the instrument under the measurement conditions described below. The measurement principle was wet laser diffraction and scattering. During the measurement, laser beams were detected according to a transmission method, and the particle size was calculated with a particle refractive index of 1.81 and an assumed sample type being non-spherical particles.
Instrument: Microtrac MT3200II (Microtrac Bell Corporation)
Measurement solvent: Isopropanol (solvent refractive index: 1.38)
Measurement time: 10 seconds

TABLE 8

| | Granules that remained in No. 60 sieve (Example 26) | Granules that passed through No. 60 sieve (Example 27) |
|---|---|---|
| Cumulative Particle Size Distribution of 10%: D10 (µm) | 85.4 | 34.5 |
| Cumulative Particle Size Distribution of 50%: D50 (µm) | 362.5 | 78.3 |
| Cumulative Particle Size Distribution of 90%: D90 (µm) | 675.6 | 182.2 |

The results of the cumulative particle size distribution of 10% (D10), cumulative particle size distribution of 50% (D50), and cumulative particle size distribution of 90% (D90) indicate that the particle size of the granules that remained in the No. 60 sieve was larger than that of the granules that passed through the No. 60 sieve, and their D50 values, which are considered to be their average particle size, differed by more than a factor of 4. However, the progress distance (D1) of the samples of Examples 26 and 27 were both shorter than that of Comparative Example 11, indicating that the reactivity between the protamine degradation product and carrageenan was reduced. The samples of Examples 26 and 27 were equal in terms of the relative degree of progress RD (the RD determined with the sample of Comparative Example 11 as a control sample), and no difference was found between Examples 26 and 27.

The results above indicated that the difference in the particle size of at least D50 within the range of 78.3 to 362.5 µm makes no difference in the effect of inhibiting the reaction between the protamine degradation product and carrageenan by shellac coating. Additionally, even though the content of the protamine degradation product was as high as 40%, shellac coating was effective in limiting the decrease in viscosity of carrageenan.

### Test Example 8: Oral Retention

Viscosity is associated with adherence in the oral cavity. The granular formulations of Example 27 and Comparative Example 11 were evaluated for oral retention. For evaluation, the intensity of the ninhydrin reaction indicated by saliva was considered as the amount of peptides derived from the protamine degradation product (the amount of the protamine degradation product residue).

Before the test, saliva was collected for use as a blank sample. Subsequently, only 100 mg of a protamine degradation product, or the granular formulation (250 mg) of Example 27 or Comparative Example 11, which was 100 mg on a protamine degradation product basis, was placed on the tongue, and saliva was collected over time while the protamine degradation product or the granular formulation was gently spread in the oral cavity.

### Measurement of Amount of Protamine Degradation Product Residue

After 500 µL of collected saliva, 5.5 mL of purified water, and 1 mL of an aqueous solution of 0.2% (w/w) ninhydrin were mixed in a test tube, the test tube was heated in hot water for 10 minutes. The test tubes were centrifuged and the supernatant was collected. The absorbance of the supernatant at 570 nm was measured, and the absorbance intensity attributed to the protamine degradation product was determined by subtracting the value indicated by the blank sample from the measured absorbance. FIG. 1 shows the results.

The sample of Example 27 and the sample of Comparative Example 11 both showed a maximum amount of the protamine degradation product residue (absorbance intensity) at 2 minutes after intraoral administration. FIG. 1 shows the absorbance intensity at this time as “1” and the absorbance intensities of the samples collected at other points of time as relative values. In the case of administration of the protamine degradation product alone, the retention time was not shorter than 5 minutes and shorter than 8 minutes because the presence of the protamine degradation product could not be confirmed in the saliva sample after 8 minutes. The retention time of the granular formulation of Example 27 was not shorter than 30 minutes and shorter than 35 minutes, and the retention time of the granular formulation of Comparative Example 11 was not shorter than 10 minutes and shorter than 12 minutes. This indicates that the shellac coating greatly extended the oral retention time of the protamine degradation product.

Additionally, as shown in Test Example 7, the decrease in viscosity was more limited in the granules of Example 27 than in the granules of Comparative Example 11. This strongly suggests that the compositions of the Examples that were confirmed to have limited the decrease in viscosity would also have an oral retention time of the functional substance longer than uncoated granules.

### Test Example 9: Change in Viscosity of Acidic Polysaccharide Due to Siberian Ginseng Extract Powder

The viscosity of an acidic polysaccharide was measured and evaluated in the same manner as in Test Example 1, except that a Siberian ginseng extract powder was used as the functional substance.

TABLE 9

| | Functional Substance | Coating Compound | Acidic Polysaccharide | D1-D0 (mm) | D2-D0 (mm) | RD (Evaluation) |
|---|---|---|---|---|---|---|
| Example 28 | Siberian Ginseng Extract Powder | Shellac | Carrageenan | 35 | 14 | 0.4 (A) |

Because the Siberian ginseng extract powder decreased the viscosity of carrageenan as in Examples 1 to 10 (D1-D0 in Examples 5 to 10: 16 to 35 mm), the Siberian ginseng extract powder was assumed to have reacted with carrageenan in the same manner as the green tea extract, cassis extract, coffee bean extract, guava extract, or blueberry extract. However, a Siberian ginseng extract powder coated with shellac showed a limited decrease in viscosity as in Examples 1 to 9, and the viscosity approached the original physical properties of carrageenan (D2-D0 in Examples 5 to 10: 2 to 7 mm). The RD was lower than 0.9, and shellac coating was able to inhibit the reaction with carrageenan and its adsorption. The inhibition of reaction and adsorption by shellac coating appeared to be able to sufficiently limit the decrease in viscosity of acidic polysaccharides.

The Siberian ginseng extract powder is rich in lignan compounds such as syringin (eleutheroside B) and eleutheroside E; coumarin compounds such as isofraxidin; and polyphenolic compounds such as chlorogenic acid. These substances appeared to have reacted with carrageenan (acidic polysaccharide).

### Test Example 10: Change in Viscosity of Acidic Polysaccharide Due to Polyphenolic Compound, Lignan Compound, or Basic Substance

In the same manner as in Test Example 2, epigallocatechin gallate (polyphenolic compound), syringin and eleutheroside E (lignan compounds), and spermidine (basic substance) were prepared, and the effects of these substances on the viscosity of carrageenan were measured, and their reactivity was evaluated. Note that spermidine was dissolved in purified water, and the prepared aqueous solution was used as a sample. Table 10 shows the results.

TABLE 10

| | | RI |
|---|---|---|
| Comparative Example 12 | Epigallocatechin Gallate | 4.4 |
| Comparative Example 13 | Syringin | 2.3 |
| Comparative Example 14 | Eleutheroside E | 2.1 |
| Comparative Example 15 | Spermidine | 1.5 |

The calculated RI of epigallocatechin gallate (polyphenolic compound), syringin, eleutheroside E (lignan compounds), and the solution of spermidine (basic substance) was 1.5 to 4.4 (uncoated samples), and these substances in such a slight amount of 10 ppm decreased the viscosity of aqueous carrageenan solutions. Epigallocatechin gallate is contained in tea leaf extracts such as green tea, black tea, and oolong tea. Syringin is abundant in Eleutherococcus senticosus, Taraxacum, Ilex rotunda, Tinospora crispa, and plants of the genus Saussurea. Eleutheroside E is abundant in Eleutherococcus senticosus. Spermidine is abundant in soybeans, adzuki beans, shiitake mushrooms, other mushrooms, bell peppers, green peas, pistachios, corn, wheat germ, blue cheese, pollack roe, salmon caviar, derma, and chicken liver. From the results of these substances, it was assumed that not only the plant extracts confirmed to have reacted with carrageenan in Test Examples 1 and 9, but also other plant extracts would react with carrageenan and decrease viscosity.

### Test Example 11: Concentration and Type of Coating Compound

D0, D1, D2, and RD were determined in the same manner as in Test Example 4, except that a green tea extract was used as a functional substance, and that the type and concentration of the coating compound were as indicated in Table 11 below. Table 11 shows the results.

TABLE 11

| | Functional Substance | Coating Compound | Acidic Polysaccharide | D1-D0 (mm) | D2-D0 (mm) | RD (Evaluation) |
|---|---|---|---|---|---|---|
| Example 29 | Green Tea Extract | Shellac (1%) | Carrageenan | 28 | 22 | 0.8 (A) |
| Example 30 | Green Tea Extract | Shellac (10%) | Carrageenan | 28 | 13 | 0.5 (A) |
| Example 31 | Green Tea Extract | Shellac (20%) | Carrageenan | 28 | 11 | 0.4 (A) |
| Example 32 | Green Tea Extract | Shellac (30%) | Carrageenan | 28 | 10 | 0.4 (A) |
| Example 33 | Green Tea Extract | Zein (10%) | Carrageenan | 28 | 3 | 0.1 (A) |

Coating with shellac in an amount of 1 mass% of the green tea extract even limited the decrease in viscosity of the aqueous carrageenan solution; the decrease in viscosity was limited dependently on the amount of shellac added (Examples 29 to 32). This indicates that the results of Test Example 4 are reproducible, and that shellac in an amount at least within the range of 1 to 30 mass% of the functional substance has an effect of inhibiting the reaction with carrageenan. Additionally, the RD was lower than 0.9 due to coating with zein (Example 34), indicating that the reaction between the green tea extract and carrageenan was inhibited. Thus, the coating effect of zein was reproduced.

### Test Example 12: Type of Acidic Polysaccharide

The effects of shellac coating on the viscosity of acidic polysaccharides were measured in the same manner as in Test Example 5, except that the conditions described in the following Table 12 were applied. Table 12 shows the results.

TABLE 12

| | Functional Substance | Coating Compound | Acidic Polysaccharide | D1-D0 (mm) | D2-D0 (mm) | RD (Evaluation) |
|---|---|---|---|---|---|---|
| Example 34 | Green Tea Extract | Shellac (15%) | Sodium Alginate | 16 | 1 | 0.1 (A) |
| Example 35 | Green Tea Extract | Shellac (15%) | Xanthan Gum | 13 | 3 | 0.3 (A) |
| Example 36 | Green Tea Extract | Shellac (15%) | Hyaluronic Acid | 10 | 2 | 0.4 (A) |

Hyaluronic acid, xanthan gum, and sodium alginate, which are acidic polysaccharides, reacted with a green tea extract and decreased the viscosity of the 1% aqueous solution. However, because shellac coating greatly limited the decrease in viscosity as in the case of carrageenan, shellac coating was found to be effective in limiting the decrease in viscosity of the acidic polysaccharides other than carrageenan and thereby effective in limiting the decrease in oral retention. The results of Test Example 5 were also reproduced.

### Test Example 13: Relationship Between Amount of Acidic Polysaccharide and Granule Hardness

Granular formulations were prepared according to the formulas shown in Table 13. The unit for the values in Table 13 is gram. A solution of 25 mass% shellac in ethanol was added dropwise to a green tea extract. The ethanol solution was added in such an amount that shellac was 15 mass% of the green tea extract. After being fully mixed, the mixture was dried in a dryer at 50° C., and the dried product was classified with a No. 60 sieve (mesh size: 250 µm) to obtain granules that passed through the sieve (coated granules). The coated granules were mixed with carrageenan, and hydrous ethanol with an ethanol concentration of 90% (v/v) in an amount of 40 mass% of the amount of carrageenan was further mixed dropwise. Thereafter, extrusion granulation was performed using a 1-mm-diameter stainless steel mesh, followed by drying in a dryer at 50° C., thereby preparing granules (Examples 37 to 40 and Comparative Example 16). Extrusion granulation was also performed in the same manner by using a green tea extract that was not coated with shellac to prepare granules (Comparative Examples 17 to 20). These granules were subjected to the granule strength test described below.

TABLE 13

| | Example 37 | Example 38 | Example 39 | Example 40 | Example 41 |
|---|---|---|---|---|---|
| Green Tea Extract | 2.5 | 1.0 | 1.0 | 1.0 | 1 |
| Shellac | 0.38 | 0.15 | 0.15 | 0.15 | 0.15 |
| Carrageenan | 2.5 | 5 | 10 | 20 | 30 |
| Carrageenan/Green Tea Extract | 1 | 5 | 10 | 20 | 30 |

Continued from TABLE 13

| | Comparative Example 16 | Comparative Example 17 | Comparative Example 18 | Comparative Example 19 | Comparative Example 20 |
|---|---|---|---|---|---|
| Green Tea Extract | 2.5 | 1 | 1 | 1 | 1 |
| Shellac | 0 | 0 | 0 | 0 | 0 |
| Carrageenan | 2.5 | 5 | 10 | 20 | 30 |
| Carrageenan/Green Tea Extract | 1 | 5 | 10 | 20 | 30 |

### Granule Strength Test

All of the prepared granules (Examples 37 to 41 and Comparative Examples 16 to 20) were sieved with a 710-µm-diameter sieve to remove powder equal to or smaller than 710 µm, and granules remaining on the sieve were collected. Five grams of the collected granules were further placed on the 710-µm-diameter sieve and forcibly impacted (intensity: 1.5 mm/g, time: 5 minutes) with an electromagnetic vibratory sieve shaker (Retsch GmbH, AS200 Control), followed by calculating the percentage of granules remaining on the sieve. In this test, because particles equal to or smaller than 710 µm were removed beforehand, a higher residual percentage means higher granule strength. Table 14 shows the results in unit of percent.

TABLE 14

| Ratio of Carrageenan/Green Tea | 1 | | 5 | | 10 | |
|---|---|---|---|---|---|---|
| Division | Comparative Example 16 | Example 37 | Comparative Example 17 | Example 38 | Comparative Example 18 | Example 39 |
| 710 µm or larger | 81.2 | 94.3 | 70 | 83.1 | 34.1 | 65 |

Continued from TABLE 14

| Ratio of Carrageenan /Green Tea | 20 | | 30 | |
|---|---|---|---|---|
| Division | Comparative Example 19 | Example 40 | Comparative Example 20 | Example 41 |
| 710 µm or larger | 4.5 | 14.8 | 4.8 | 6.4 |

The results of Comparative Examples 16 to 20 reveal that the higher the ratio of carrageenan added, the lower the residual percentage, likely resulting in brittle granules. However, Examples 37 to 41 showed higher residual percentages, indicating limited decreases in granule strength. This test revealed that shellac coating increases the strength of granules prepared with an acidic polysaccharide. This phenomenon had never been reported before and is a new finding. If the granule strength is low, the granules would crumble due to vibration during the manufacturing process, and the resulting fine powders would enter machinery or packaging materials, causing manufacturing problems. Thus, higher strength is desirable. In view of this experiment, the amount of the acidic polysaccharide for use is preferably 1 to 30 times the amount of the functional substance, and more preferably 1 to 20 times the amount of the functional substance.

### Test Example 14: Oral Retention Test Using Green Tea Extract

Table 15 shows the formulas of tablets with a unit weight of 1000 mg. In Comparative Example 21 and Example 42, tablets were prepared with a total batch weight of 200 g. In Comparative Example 21, the raw materials were mixed and tableted with a rotary tableting machine (Kikusui Corporation, Clean Press 19 K) with a pestle (15 mm in diameter) attached (tablet weight: 1000 mg, rotary speed: 35 rpm). The hardness of the resulting tablets was 10 kgf or more. In Example 42, a solution of zein in ethanol (aqueous solution of 80% ethanol) was added dropwise to a green tea extract and lactitol, and the mixture was stirred with a high-speed mixer (Fukae Pautech, LFS-GS-1J) at an agitator speed of 300 rpm and a chopper speed of 1200 rpm to homogeneously mix the materials. Thereafter, the mixture was dried with warm air at a temperature of room temperature to 90° C. with a fluidized bed granulation dryer (Freund Corporation, FLO-5A) and crushed with a household mill, followed by classification with a No. 30 sieve (mesh size: 600 µm), thereby obtaining granules that passed through the sieve (coated granules). Carrageenan, HPMC, sorbitol, fine silicon dioxide, and calcium stearate were added to the classified granules and mixed, thereby obtaining powder for tablets. The obtained powder was tableted in the same manner as in Comparative Example 21, thereby obtaining tablets with a hardness of 10 kgf or more.

TABLE 15

| | Comparative Example 21 | Example 42 | Example 43 |
|---|---|---|---|
| Green Tea Extract | 28.6 mg | 28. 6 mg | 28.6 mg |
| Lactitol | 20 mg | 20 mg | 20 mg |
| Zein | | 2.86 mg | 2.86 mg |
| Carrageenan | | 80 mg | 40 mg |
| HPMC | | 80 mg | 40 mg |
| Sorbitol | 911.4 mg | 748.54 mg | 828.54 mg |
| Fine Silicon Dioxide | 20 mg | 20 mg | 20 mg |
| Calcium Stearate | 20 mg | 20 mg | 20 mg |
| Total | 1000 mg | 1000 mg | 1000 mg |

### Oral Retention Test

This test was conducted with volunteered three normal healthy subjects. First, the subjects took water in the mouth and gargled three times beforehand, and then their saliva was collected at the start of the test (0 minutes) as a blank sample. Subsequently, the subjects took the tablet of either Comparative Example 21 or Example 42 in the mouth and chewed within 1 minute, followed by sampling saliva after 10, 20, and 30 minutes. To evaluate the green tea extract in the saliva, a 1,1-diphenyl-2-picrylhydrazyl (DPPH) radical scavenging activity assay was performed to examine the persistence of the green tea extract in the oral cavity. In this test, saliva at 0 minutes had no DPPH radical scavenging activity, and the green tea extract has DPPH radical scavenging activity. Thus, collected saliva that has DPPH radical scavenging activity indicates the green tea extract remaining in the saliva.

### DPPH Radical Scavenging Activity Assay

First, a solution of 1 mM DPPH in methanol was prepared in the dark. This solution was diluted with 50% acetonitrile to achieve an absorbance of 0.65 at 517 nm. 25 µl of 50% acetonitrile was added to 1475 µl of the solution of 1 mM DPPH in methanol, and the mixed solution was determined to be a blank solution. Subsequently, 100 µl of saliva collected as a sample and 100 µl of acetonitrile were mixed with a vortex mixer. Thereafter, sonication was performed for about 30 seconds, followed by centrifugation at 5000 G for 5 minutes to obtain 25 µl of the supernatant as a sample solution, which was then mixed with 1475 µl of another solution of 1 mM DPPH in methanol diluted as above with a vortex mixer. After mixing, the mixture was allowed to react in the dark for 30 minutes, and the absorbance at 517 nm was measured with a spectrophotometer (U-2900, Hitachi High-Tech Science Corporation). The radical scavenging capacity was calculated according to the following formula.

$$\begin{matrix}R a d i c a l S c a v e n g i n g C a p a c t i y \left(\%\right) = \\ \frac{O . D 517 \left(B l a n k S o l u t i o n\right) - O . D 517 \left(S a m p l e S o l u t i o n\right)}{O . D 517 \left(B l a n k S o l u t i o n\right) \times 100} \times 100\end{matrix}$$

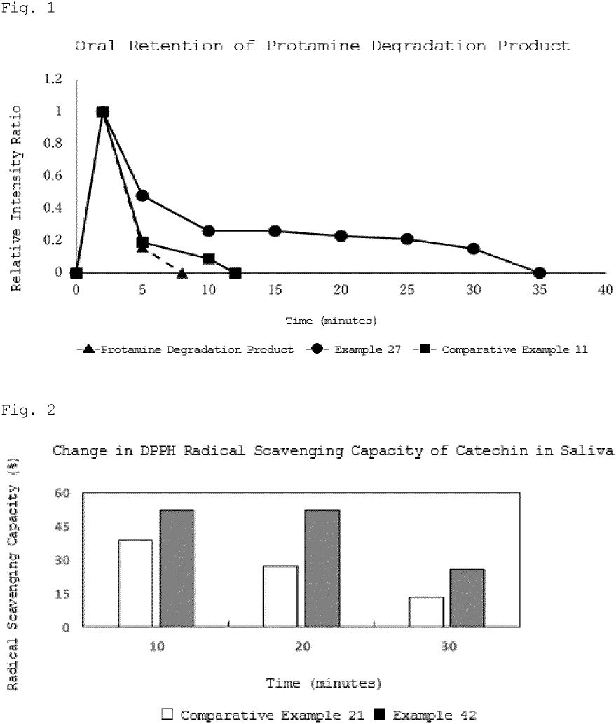

FIG. 2 shows the results. A comparison between Comparative Example 21 and Example 42 indicates that given the higher radical scavenging capacity, the green tea extract remained more in the oral cavity in Example 42, showing a higher retention performance in Example 42 than in Comparative Example 21. Example 42 showed a robust retention in the oral cavity even after 30 minutes.

### Test Example 15: Study 1 of Bad Breath Control Using Green Tea Extract

The effects of the tablets of Comparative Example 21 and Example 42 on bad breath control were examined. The total amount of volatile sulfur compounds in exhaled breath was quantitatively analyzed with an XP-Breath-Tron (New Cosmos Electric Co., Ltd.) to evaluate the changes in bad breath. The following describes the experiment.

### Experimental Study of Bad Breath Control

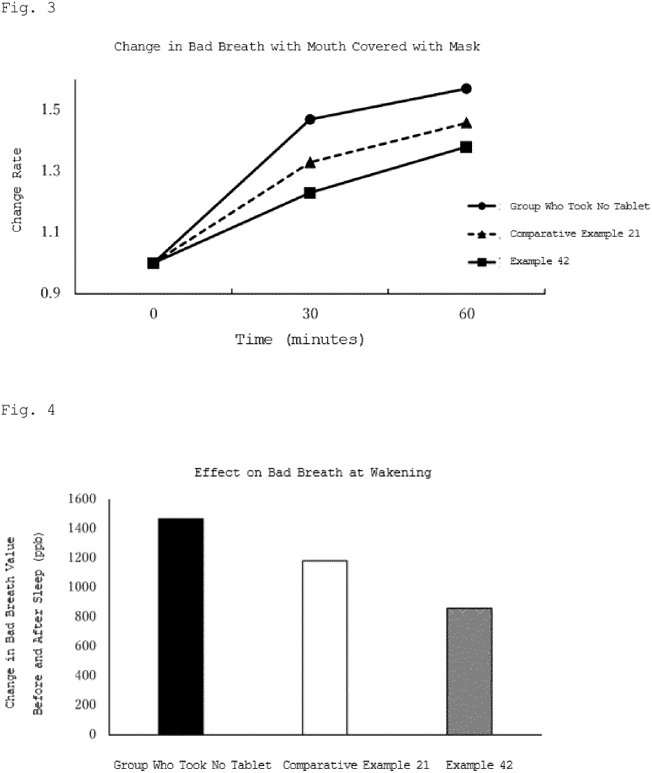

Five normal healthy subjects (volunteers) were not allowed to consume garlic-containing foods the day before, and were not allowed to consume palate-pleasing items such as coffee and tea, and not allowed to smoke tobacco, before the test on the day of the test. First, the subjects took water in the mouth and gargled three times beforehand, and then wore a mask. The changes in bad breath over a period of one hour were measured. Thereafter, they placed the tablet of Comparative Example 21 or Example 42 in their mouth, chewed it within a minute, and were allowed to spend time as usual wearing a mask. After ingestion of the test substance, the subjects were not allowed to eat and drink, and volatile sulfur compounds in the exhaled breath were quantified every 30 minutes. FIG. 3 shows the results.

The rate of change was higher in the group of subjects who did not take the tablet, indicating a worsening bad breath over time while the subjects wore a mask. Whereas the intake of the tablet of Comparative Example 21 slightly inhibited bad breath from worsening, the intake of the tablet of Example 42 further inhibited bad breath from worsening. As noted above, the green tea extract in the tablet of Example 42 was retained in the oral cavity; due to this retention of the green tea extract, the tablet of Example 42 appeared to have inhibited bad breath from worsening.

### Test Example 16: Study 2 of Bad Breath Control Using Green Tea Extract

The tablets of Comparative Example 21 and Example 42 were used to examine their effects in inhibiting bad breath on waking. To evaluate bad breath, the total amount of volatile sulfur compounds in exhaled breath was quantitatively analyzed with an XP-Breath-Tron (New Cosmos Electric Co., Ltd.). The following describes the experiment.

### Experimental Study of Bad Breath Control

One normal healthy subject (volunteer) was not allowed to consume garlic-containing foods and palate-pleasing items such as coffee and tea in the evening of the day for experiment. The subject brushed teeth one hour before bedtime. After one hour, just before bedtime, volatile sulfur compounds were measured. The subject slept for six hours and woke up, and was immediately measured for the volatile sulfur compounds. In the same manner, the volatile sulfur compounds before bedtime were measured, and the subject took the tablet of either Comparative Example 21 or Example 42. The subject then chewed it within one minute and slept for six hours. After the subject woke up, volatile sulfur compounds were measured. FIG. 4 shows the results.

The results of the group who did not take the tablets indicate the degree of deterioration of bad breath before and after bedtime. Whereas the intake of the tablet of Comparative Example 21 slightly inhibited bad breath from worsening, the intake of the tablet of Example 42 further inhibited bad breath on waking from worsening. As noted above, the green tea extract in the tablet of Example 42 was retained in the oral cavity; the further enhanced retention of the green tea extract due to a very low saliva volume during sleep appears to have inhibited bad breath from worsening for a long period of time.

### Test Example 17: Sweetness Persistence Test Using Sweetener

Table 16 shows the formulas of granules with a unit weight of 100 mg. Based on these formulas, the granules of Examples 43, 44, and 45 each in a total weight of 200 g were prepared. A solution of zein in ethanol (aqueous solution of 80% ethanol) was added dropwise to sucralose, aspartame, and xylitol individually and mixed homogeneously with stirring with a high-speed mixer at an agitator speed of 300 rpm and a chopper speed of 1200 rpm. The granules were then dried with warm air at a temperature of room temperature to 90° C. with a fluidized bed granulation dryer (Freund Corporation, FLO-5A) and crushed with a household mill, followed by classification with a No. 60 sieve (mesh size: 250 µm), thereby obtaining granules that passed through the sieve (coated granules). Carrageenan and HPMC were added to the classified granules and homogeneously mixed with stirring with the same high-speed mixer as above. Thereafter, the mixture was then dried with warm air at a temperature of room temperature to 90° C. in a fluidized bed granulation dryer (Freund Sangyo, FLO-5A) and crushed with a household mill, followed by classification with a No. 60 sieve (mesh size: 250 µm), thereby obtaining powder that passed through the sieve. The obtained granules were subjected to the sensory test described below. The sweeteners alone were also subjected to the sensory test as Comparative Examples 22, 23, and 24.

TABLE 16

| | Example 43 | Example 44 | Example 45 | Comparative Example 22 | Comparative Example 23 | Comparative Example 24 |
|---|---|---|---|---|---|---|
| Sucralose | 89 mg | | | 100 mg | | |
| Aspartame | | 80 mg | | | 100 mg | |
| Xylitol | | | 80 mg | | | 100 mg |
| Zein | 3 mg | 8 mg | | | | |
| Carrageenan | 4 mg | 4 mg | 4 mg | | | |
| HPMC | 4 mg | 8 mg | 8 mg | | | |
| Total | 100 mg | 100 mg | 100 mg | 100 mg | 100 mg | 100 mg |

### Sensory Test

Five normal healthy subjects (volunteers) were not allowed to consume palate-pleasing items such as coffee and tea, and not allowed to smoke tobacco, before the test on the day of the test, and then the test was performed. The granules containing sucralose or aspartame of Examples 43 and 44, and the sweeteners of Comparative Examples 22 and 23 were each placed on the tongue in such an amount that the intake of sucralose or aspartame was 20 mg. The granules containing xylitol of Example 45 and the sweetener of Comparative Example 24 were each placed on the tongue in such an amount that the intake of xylitol was 100 mg. After ingestion, the subjects spent time as usual, during which they were not allowed to eat and drink, and then asked to self-report the point in time at which they no longer perceived a sweet taste. The time was recorded. Table 17 shows the results.

As shown in Table 17, all of the Examples exhibited longer perception of sweetness than the Comparative Examples, which were the sweeteners themselves. The coating of the granules of Examples 43 to 45 with zein (coating agent) and the longer retention in the oral cavity due to carrageenan appeared to have prolonged sweet taste perception. The difference in the time of persistence is thought to be due to the threshold of sweetness exhibited by each sweetener. For example, the threshold for sucralose is reported as 0.2 mg/L. The lower the taste threshold, the more sustained the perception of sweetness would be. The same effects are thought to be achieved in other sweeteners, such as somatin, curculin, monellin, monatin, miraculin, stevia, glycyrrhizin, neotame, advantame, acesulfame potassium, and saccharin, and there also appears to be a lasting effect of tastes other than sweetness, such as saltiness, pungency, sourness, and umami.

TABLE 17

| | Sucralose | | Aspartame | | Xylitol | |
|---|---|---|---|---|---|---|
| | Comparative Example 22 | Example 43 | Comparative Example 23 | Example 44 | Comparative Example 24 | Example 45 |
| Average time during which taste was perceived | 3 min 28 sec | 7 min 17 sec | 3 min 16 sec | 5 min 1 sec | 28 sec | 37 sec |

### Test Example 18

The tablets of Example 42 and Comparative Example 21 were used to evaluate the intensity of bitterness. The evaluation method is described below.

### Evaluation Method for Bitterness

Five normal healthy subjects (volunteers) were not allowed to consume palate-pleasing items such as coffee and tea, and not allowed to smoke tobacco, before the test on the day of the test. After gargling with water beforehand, the subjects placed a sample into the mouth, chewed, and evaluated the intensity of bitterness sensorially. The samples for use were a single tablet of Example 42 (1000 mg, which corresponds to 28.6 mg of a green tea extract), a single tablet of Comparative Example 21 (1000 mg, which corresponds to 28.6 mg of a green tea extract), and a green tea extract (28.6 mg of powder). Each subject tested all of the samples and scored each sample. The test was performed in the following order: the tablet of Example 42, the tablet of Comparative Example 21, and the green tea extract. The criteria for scoring were as given below. The average of the scores of the five subjects was calculated.
1: Hardly bitter
2: Slightly bitter
3: Bitter
4: Very bitter
5: Strongly bitter

Table 18 shows the results. The score of Comparative Example 21 was lower than that of the green tea extract raw material itself. This appeared to be due to masking of bitterness with sweetness of the sorbitol (excipient) used to form the tablet. However, the score of Example 42 was even lower, at half the score of the green tea extract raw material itself. This indicates that the sample of Example 42 had a masking effect on bitterness. Although this test was performed for evaluation of the masking of bitterness, masking of saltiness, sourness, unpleasant bitterness (egumi), and pungency would also be possible.

TABLE 18

| | Comparative Example 21 | Example 42 | Green Tea Extract |
|---|---|---|---|
| Scoring for Bitterness | 3.8 | 2.5 | 5.0 |

### Test Example 19: Production Example of Chewable Tablet (Example 46)

Tablets were produced in the same manner as the tablets of Example 42, except that the amount of raw material components for use was as listed in Table 19, and that the green tea extract and lactitol were replaced with L8020 lactic acid bacteria powder and xylitol, respectively. The resulting tablets had a hardness of 10 kgf or higher, which was a sufficient hardness. No problems were also found in manufacturability.

TABLE 19

| | Example 46 |
|---|---|
| L8020 Lactic Acid Bacteria | |
| Powder | 37.5 mg |
| Xylitol | 217.5 mg⁵ |
| Zein | 25.5 mg |
| Carrageenan | 45 mg |
| HPMC | 40 mg |
| Sorbitol | 594.5 mg |
| Fine Silicon Dioxide | 20 mg 10 |
| Calcium Stearate | 20 mg |
| Total | 1000 mg |

1. An oral retention composition comprising at least one compound selected from the group consisting of shellac and zein, a functional substance, and an acidic polysaccharide, the functional substance coated with the at least one compound selected from the group consisting of shellac and zein being mixed with the acidic polysaccharide.

2. The oral retention composition according to claim 1, wherein the acidic polysaccharide is at least one member selected from the group consisting of carrageenan, hyaluronic acid, xanthan gum, sodium alginate, pectin, and gum arabic.

3. The oral retention composition according to claim 1, wherein the functional substance is at least one member selected from the group consisting of a basic substance, a polyphenol, and a lactic acid bacterium.

4. The oral retention composition according to claim 3, wherein the basic substance has an amino group, and the polyphenol has two or more hydroxyl groups bonded to an aromatic ring.

5. The oral retention composition according to claim 3, wherein the basic substance is at least one member selected from the group consisting of a protamine degradation product, arginine, lysine, glucosamine, spermine, spermidine, putrescine, piperine, and chitosan.

6. The oral retention composition according to claim 3, wherein the polyphenol is at least one member selected from the group consisting of chlorogenic acid, quercetin, resveratrol, tannic acid, caffeic acid, caffeic acid phenethyl ester, hydroxytyrosol, oleuropein, gallic acid, eugeniin, catechin, daidzein, procyanidin, theaflavin, polyphenol-containing plant extract, propolis, champignon extract, syringin, and eleutheroside E.

7. The oral retention composition according to claim 1, which is in the form of granules, powder, or tablets.

8. The oral retention composition according to claim 1, which has an average particle size of 1 µm to 400 µm.

9. The oral retention composition according to claim 1, which is a material for producing a composition for oral cavity or an oral composition.

10. A composition for oral cavity or an oral composition, comprising the oral retention composition according to claim 1.

11. The composition for oral cavity or oral composition according to claim 10, which is a food, an oral care product, a pet food, a drug, or a quasi-drug.

12. The composition for oral cavity or oral composition according to claim 11, which is a food for bad breath control.

13. The composition for oral cavity or oral composition according to claim 12, which is a food for bad breath control while wearing a mask or a food for bad breath control at the time of awakening.

14. The composition for oral cavity or oral composition according to claim 11, which is a taste retention food or a taste-masking food.

15. A method for producing an oral retention composition comprising at least one compound selected from the group consisting of shellac and zein, a functional substance, and an acidic polysaccharide, the functional substance coated with the at least one compound selected from the group consisting of shellac and zein being mixed with the acidic polysaccharide;
the method comprising:
step 1 of mixing the at least one compound selected from the group consisting of shellac and zein dissolved or dispersed in a solvent with the functional substance so that the compound coats the functional substance; and
step 2 of mixing the functional substance coated with the at least one compound selected from the group consisting of shellac and zein obtained in step 1 with the acidic polysaccharide.

16. The production method according to claim 15, wherein in step 2, the functional substance is mixed with the acidic polysaccharide while ethanol or hydrous ethanol with an ethanol concentration of 50% to 99% (v/v) is added.
